(19) United States
(12) Patent Application Publication
(10) Pub. No.: US 2024/0299022 A1
(43) Pub. Date: Sep. 12, 2024

(54) EYE BAG SUPPORT

(71) Applicant: Naoteru NISHITANI, Tokyo (JP)

(72) Inventor: Naoteru NISHITANI, Tokyo (JP)

(21) Appl. No.: 18/573,568

(22) PCT Filed: Jun. 28, 2022
(86) PCT No.: PCT/JP2022/025707

§ 371 Date: Dec. 22, 2023

(30) Foreign Application Priority Data
Jun. 28, 2021 (JP) 2021-107139

(51) Int. Cl. A61B 17/02; A61B 17/00

(52) U.S. Cl. CPC A61B 17/0231; A61B 2017/00738

(57) ABSTRACT

To provide a support device capable of easily supporting a lower eyelid (eye bag) while being grasped with a hand. The present invention provides a T-shaped support for upwardly supporting an eye bag of an eye, the support including a horizontal receiving part (1) to be brought into contact with the eye bag and a grip part (2) to be grasped, the grip part (2) being formed to hang from the center of the horizontal receiving part (1). The horizontal receiving part (1) is formed in a crescent shape and has both ends each formed into a curve. Further, the horizontal receiving part (1) and the grip part (2) are each formed of a metal, and the horizontal receiving part (1) is coated with silicone resin.

### TECHNICAL FIELD

The present invention relates to a support that supports a lower eyelid from below, and more specifically to a support that supports the skin of a lower eyelid from below when an eye bag is formed in a cosmetic plastic surgical procedure.

### BACKGROUND ART

In recent years, a cosmetic plastic surgical procedure has been widely performed among young people, particularly young women.

Among them, cosmetic surgery, which can be relatively easily performed, such as so-called minor cosmetic surgery has become popular.

Particularly, cosmetic surgery of an eye as a portion that brings out facial attractiveness has been popular with women.

Although examples of the eye cosmetic surgery include various types of surgery such as double eyelid formation and glamorous line formation, one of them is a procedure for forming an eye bag under an eye.

This procedure is one for injecting a filler such as hyaluronic acid into a portion of a lower eyelid to make the eye bag clear or newly form an eye bag itself.

When this surgery is performed, an operation for supporting a portion of the eye bag as the portion of the lower eyelid from below is required when giving the injection.

If this operation is not performed, the eye bag thickens, resulting in an unnatural state.

Therefore, a portion of an eye bag has been conventionally supported with fingers of a hand. However, the fingers of the hand are relatively flexible, whereby the eye bag cannot necessarily be accurately supported.

Thus, a simple device, e.g., a device capable of easily supporting an eye bag in a lower eyelid from below by being grasped with a hand has been desired.

However, unfortunately, such a device capable of supporting an eye bag has not yet been developed.

Note that during double eyelid formation, a pasting utensil as a device that grasps a forming tape has been used (see PTL 1).

During cataract surgery, an eyeball fastener for temporarily fastening an eyeball has been used (see PTL 2).

Thus, for an eye bag, a device that supports the eye bag has also been strongly desired.

### CITATION LIST

### Patent Literature

PTL 1: Japanese Patent Application Laid-Open No. 2018-51104

PTL 2: Japanese Patent Application Laid-Open No. 2020-18839

### SUMMARY OF INVENTION

### Technical Problem

The present invention has been developed upon receipt of the above-described problem.

That is, an object is to provide a support device capable of easily supporting a lower eyelid (eye bag) while being grasped with a hand.

### Solution to Problems

As a result of earnest investigations, the inventors have found that an eye bag can be easily supported by providing a horizontal support part capable of supporting a lower eyelid in a predetermined range at the tip of a grip part 2 as a part to be grasped in a support device.

The present invention is based on this finding.

That is, the present invention provides (1) a T-shaped support for upwardly supporting an eye bag of an eye, the eye bag support A including a horizontal receiving part 1 to be brought into contact with the eye bag and a grip part 2 to be grasped, the grip part 2 being formed to hang from the center of the horizontal receiving part 1.

The present invention resides in (2) the eye bag support A in the foregoing (1), in which the horizontal receiving part 1 is formed in a crescent shape and has both ends 1C each formed into a curve.

The present invention resides in (3) the eye bag support A described in the foregoing (1), in which the horizontal receiving part 1 and the grip part 2 are each formed of a metal, and the horizontal receiving part 1 is coated with silicone resin.

The present invention resides in (4) the eye bag support A described in the foregoing (1), in which the grip part 2 includes a neck part 2A having a neck inclined part 21 inclined rearward with respect to an axis provided in its upper portion, and a degree of inclination 02 of the neck inclined part 21 is 120 degrees to 170 degrees.

The present invention resides in (5) the eye bag support A described in the foregoing (4), in which the horizontal receiving part 1 is inclined forward with respect to the neck inclined part 21 to form an inclined surface 11, and a degree of inclination 01 of the inclined surface 11 is 110 degrees to 160 degrees.

The present invention resides in (6) the eye bag support A described in the foregoing (1), in which the grip part 2 has a rod shape and has fine irregularities for slippage prevention formed therein.

The present invention resides in (7) the eye bag support A described in the foregoing (5), in which the neck part 2A is provided with two angle adjustment parts M vertically spaced apart from each other, and the degree of inclination θ1 of the inclined surface 11 and the degree of inclination θ2 of the neck inclined part 21 can each be changed.

A configuration as an appropriate combination of the foregoing (1) to (7) is also possible if it meets an object of the present invention.

### Advantageous Effects of Invention

The present invention provides (1) a T-shaped support for upwardly supporting an eye bag of an eye, the support including a horizontal receiving part 1 to be brought into contact with the eye bag and a grip part 2 to be grasped, the grip part 2 being formed to hang from the center of the horizontal receiving part 1. Accordingly, the eye bag can be supported by grasping the grip part 2 and bringing the horizontal receiving part 1 into contact with the eye bag.

In the present invention, (2) the horizontal receiving part 1 is formed in a crescent shape and has both ends 1C each formed into a curve. Accordingly, the eye bag can be effectively supported.

In the present invention, (3) the horizontal receiving part 1 and the grip part 2 are each formed of a metal, and the horizontal receiving part 1 is coated with silicone resin.

Accordingly, the horizontal receiving part 1 can softly contact a skin surface when supporting the eye bag.

In the present invention, (4) the grip part 2 includes a neck part 2A having a neck inclined part 21 inclined rearward with respect to an axis provided in its upper portion, and a degree of inclination θ2 of the neck inclined part 21 is 120 degrees to 170 degrees. Accordingly, the grip part 2 does not inadvertently contact a skin surface of a face with the eye bag supported.

In the present invention, (5) the horizontal receiving part is inclined forward with respect to the neck inclined part to form an inclined surface, and a degree of inclination θ1 of the inclined surface is 110 degrees to 160 degrees. Accordingly, the eye bag can be accurately pushed up.

In the present invention, (6) the grip part 2 has a rod shape and has fine irregularities for slippage prevention formed therein. Accordingly, the grip part 2 can be accurately grasped without fingers slipping when grasped.

In the present invention, (7) the neck part 2A is provided with two angle adjustment parts M vertically spaced apart from each other, and the degree of inclination θ1 of the inclined surface 11 and the degree of inclination θ2 of the neck inclined part 21 can each be changed. Accordingly, the degree of inclination θ1 and the degree of inclination θ2 can each be adjusted, whereby a degree of freedom of a shape increases, resulting in an eye bag support A accurate for each patient.

### BRIEF DESCRIPTION OF DRAWINGS

FIG. 1 is a perspective view illustrating an eye bag support according to an embodiment of the present invention.

FIG. 2 is a diagram illustrating a horizontal receiving part in the eye bag support according to the embodiment of the present invention in an enlarged manner, where FIG. 2(A) is a front view and FIG. 2(B) is a rear view.

FIG. 3 is a side view illustrating the horizontal receiving part in the eye bag support according to the embodiment of the present invention in an enlarged manner.

FIG. 4 is a diagram illustrating an example of a manner of coating with silicone resin in the horizontal receiving part, where FIG. 4(A) illustrates an example in which silicone resin is formed in the entire horizontal receiving part, FIG. 4(B) illustrates an example in which silicone resin is formed in a portion of the horizontal receiving part, and FIG. 4(C) illustrates an example in which silicone resin is not formed for reference.

FIG. 5 is a diagram illustrating a relationship between the eye bag support and a facial surface (a chain line), where FIG. 5(A) is a diagram illustrating a state where the horizontal receiving part has not yet been brought into contact with the facial surface, and FIG. 5(B) is a diagram illustrating a state where the horizontal receiving part is brought into contact with the bottom of a lower eyelid, i.e., an eye bag and is slightly pushed up.

FIG. 6 is a front view illustrating a state leading from (A) to (B) in FIG. 5.

FIG. 7 is a diagram illustrating a curved state of a horizontal receiving part, where FIG. 7(A) illustrates a horizontal receiving part in an eye bag support according to a second embodiment, and FIG. 7(B) illustrates a horizontal receiving part in an eye bag support according to a first embodiment.

FIG. 8 is a front view illustrating an eye bag support according to a third embodiment of the present invention.

FIG. 9 is a front view illustrating an eye bag support according to a fourth embodiment of the present invention.

FIG. 10 is a side view illustrating an eye bag support A according to a fifth embodiment of the present invention.

FIG. 11 is a side view illustrating an eye bag support A according to a sixth embodiment of the present invention.

### DESCRIPTION OF EMBODIMENTS

Preferred embodiments of the present invention will be described in detail below with reference to the drawings, as needed.

In the drawings, the same elements are respectively assigned the same reference numerals, and overlapping descriptions will not be repeated.

A positional relationship among the top, the bottom, the left, and the right, for example, is based on a positional relationship illustrated in the drawings unless otherwise noted.

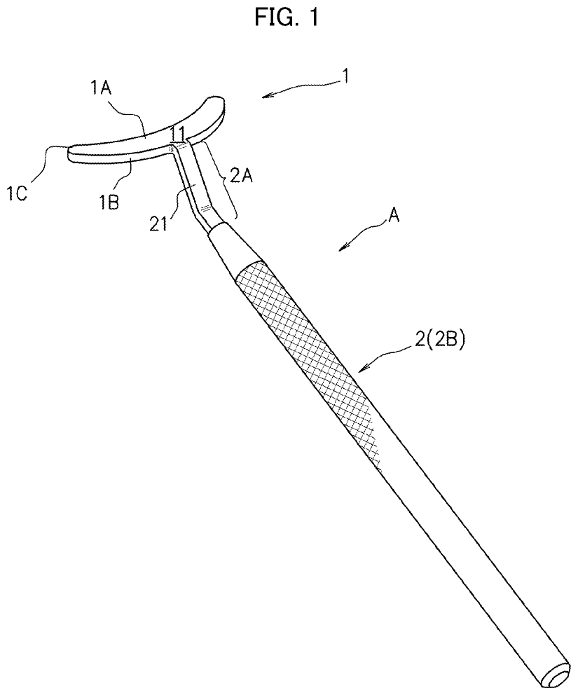

FIG. 1 is a perspective view illustrating an eye bag support A according to an embodiment of the present invention.

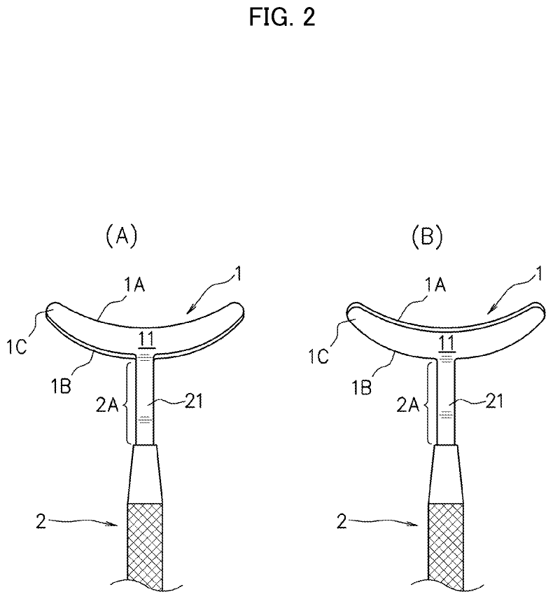

FIG. 2 is a diagram illustrating a horizontal receiving part 1 in the eye bag support A according to the embodiment of the present invention in an enlarged manner, where FIG. 2(A) is a front view and FIG. 2(B) is a rear view.

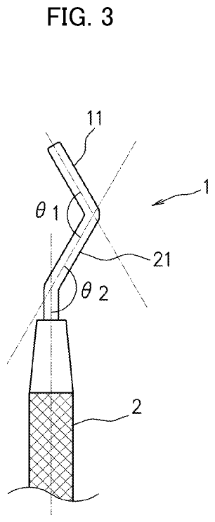

FIG. 3 is a side view illustrating the horizontal receiving part 1 in the eye bag support A according to the embodiment of the present invention in an enlarged manner.

The eye bag support A according to the present invention is a support formed in a T shape for upwardly supporting a lower eyelid, specifically, an eye bag of a face.

The eye bag support A includes the horizontal receiving part 1 to be brought into contact with the eye bag to support the eye bag and a grip part 2 to be grasped with a hand.

The grip part 2 is formed to hang from the center of the horizontal receiving part 1.

The eye bag support A enables the eye bag to be supported from below by bringing the horizontal receiving part 1 into contact with the eye bag with the grip part 2 grasped.

### (Horizontal Receiving Part 1)

The horizontal receiving part 1 has a function of supporting a skin surface slightly obliquely upwardly under predetermined pressure by contacting the bottom of the eye bag when operated.

The horizontal receiving part 1 is formed in a substantially crescent shape.

That is, the horizontal receiving part 1 is surrounded by an upper side arc 1A and a lower side arc 1B, and each of their end portions (both ends) 1C has no corner and is formed into a curve.

The horizontal receiving part 1 has a predetermined planar area between the upper side arc 1A and the lower side arc 1B, and can thus evenly contact the skin of the lower eyelid.

Therefore, the horizontal receiving part 1 can effectively support the eye bag.

The radius of curvature of the upper side arc 1A is preferably larger than the radius of curvature of the lower side arc 1B.

Since the radius of curvature of the upper side arc 1A is larger than the radius of curvature of the lower side arc 1B, the center of the horizontal receiving part 1 has the largest width.

When the eye bag support A is grasped and the horizontal receiving part 1 is brought into contact with the bottom of the eye bag in the lower eyelid and is pressed upward as it is, the skin that contacts a region between the upper side arc 1A and the lower side arc 1B is slightly pressed. As a result, the eye bag is supported by the upper side arc 1A.

The horizontal receiving part 1 is inclined forward with respect to a neck inclined part 21, described below, to form an inclined surface 11 in a side view.

Therefore, when the grip part 2 is grasped and moved, the eye bag is easily pushed up.

A degree of inclination θ1 of the inclined surface 11 in the horizontal receiving part 1 is preferably 110 degrees to 160 degrees (an angle θ1 formed with respect to the neck inclined part 21) from the viewpoint of a degree of pressing onto a facial surface in a case where the grip part 2 is grasped (see FIG. 3).

### (Grip Part)

On the other hand, the grip part 2 is formed to hang from the center of the horizontal receiving part 1.

The grip part 2 is a part to be grasped with a hand, specifically, grasped with fingers when the eye bag support A is operated.

The grip part 2 includes a neck part 2A, and includes the neck inclined part 21 inclined rearward with respect to an axis in an upper portion of the neck part 2A.

In other words, the neck part 2A includes the neck inclined part 21 inclined forward over a predetermined region.

The neck part 2A is thinner than others (i.e., than a grip body) and is formed in a flat plate shape.

Due to the presence of the neck inclined part 21, when the grip part 2 is grasped and the horizontal receiving part 1 is brought into contact with the eye bag, a predetermined distance is maintained between a lower portion of the grip part 2 and the facial surface (skin surface). As a result, the lower portion of the grip part 2 is prevented from contacting the facial surface.

A degree of inclination θ2 of the neck inclined part 21 in the grip part 2 is preferably 120 degrees to 170 degrees (an angle θ2 formed with respect to a grip body 2B) from the viewpoint of a contact property of the grip part 2 with the facial surface in a case where the grip part 2 is grasped (see FIG. 3).

The rod-shaped grip body is circular in cross section, while the neck part 2A is rectangular in cross section.

The neck part 2A is a part that easily contacts the skin when the eye bag in the lower eyelid is pushed up, and is therefore preferably a flat surface having a large area.

The grip body is a part to be grasped and operated by a person during surgery, and needs to be able to be integrated with fingers and accurately grasped.

Therefore, the grip body has fine irregularities (a knurling, etc.) for slippage prevention formed therein, and is less slippery because its friction with the fingers increases.

In the eye bag support A according to the present invention, a material therefor is a material that can withstand sterilization, and the horizontal receiving part 1 and the grip part 2 are each preferably formed of a metal (e.g., stainless steel or titanium).

The horizontal receiving part 1 formed of a metal is a part that contacts the lower eyelid of the eye. Therefore, preferably, the horizontal receiving part 1 uses a metal (e.g., stainless steel or titanium) having a smooth surface or is coated with synthetic resin (e.g., silicone resin).

When the horizontal receiving part 1 supports the eye bag, the horizontal receiving part 1 softly contacts the skin not to damage the skin surface of the eye bag because a metal having a smooth surface does not have fine irregularities and silicone resin has softness.

When the horizontal receiving part 1 is coated with silicone resin, cooling of a metal is not sensuously felt from the viewpoint of thermal conductivity.

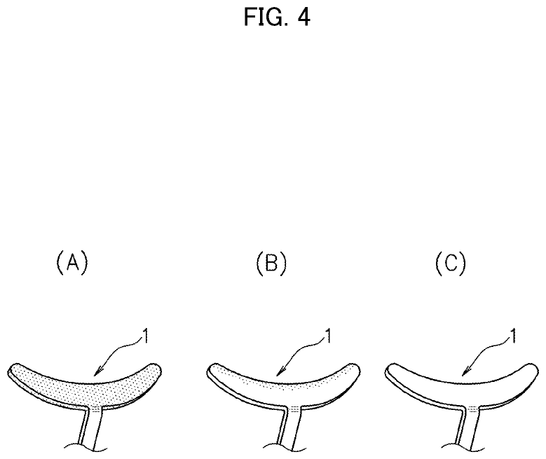

There are various manners of coating with silicone resin. FIG. 4 is a diagram illustrating examples of manners of coating with silicone resin in the horizontal receiving part.

For example, FIG. 4(A) illustrates examples in which silicone resin is formed in the entire horizontal receiving part 1.

FIG. 4(B) illustrates an example in which silicone resin is formed in a portion of the horizontal receiving part 1.

FIG. 4(C) illustrates an example in which silicone resin is not formed for reference.

### (Method for Operating Eye Bag Support A)

Then, a method for operating the eye bag support A according to the present invention will be described.

As illustrated in the figure, to operate the eye bag support A, the grip part 2 is first grasped with fingers with the horizontal receiving part 1 on the upper side.

At this time, the horizontal receiving part 1 is opposed to a facial surface.

Then, the grip part 2 is moved, and the horizontal receiving part 1 is brought into contact with the bottom of a lower eyelid, i.e., an eye bag.

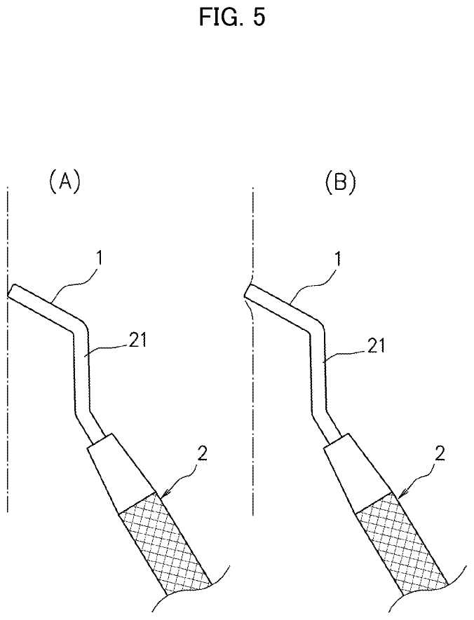

FIG. 5 is a diagram illustrating a relationship between the eye bag support A and the facial surface, where FIG. 5(A) is a diagram illustrating a state where the horizontal receiving part 1 has not yet been brought into contact with the facial surface, and FIG. 5(B) is a diagram illustrating a state where the horizontal receiving part 1 is brought into contact with the bottom of the lower eyelid, i.e., the eye bag and is slightly pushed up. They are seen in a side view.

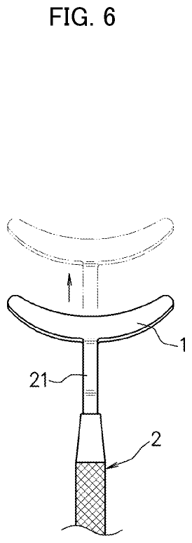

FIG. 6 is a front view illustrating a state leading from (A) to (B) in FIG. 5.

Then, the horizontal receiving part 1 is pressed against the skin as it is.

The skin receives pressure by an area portion between the upper side arc 1A and the lower side arc 1B in the horizontal receiving part 1, and the eye bag (specifically, the bottom of the eye bag) is pressed as it is by the horizontal receiving part 1.

In the case, the grip part 2 is slightly swung in an up-down direction, or the grip part 2 is turned in an axial direction, thereby making it possible to adjust an angle of pressing pressure while changing an angle of the horizontal receiving part 1 with respect to a skin surface.

The horizontal receiving part 1 easily pushes up the eye bag because it is inclined (by θ1) forward in a side view.

The neck part 2A includes the neck inclined part 21 inclined rearward with respect to an axis over a predetermined region. Accordingly, a hand with which the grip part 2 is grasped is positioned along the facial surface so that an accurate operation can be performed.

Each of both the ends 1C of the horizontal receiving part 1 has no corner and is formed into a curve, not to sharply cut into the skin.

Note that in the above-described state, surgery may be performed by injecting hyaluronic acid (e.g., 0.1 cc to 0.5 cc) in a width of 5 mm to 8 mm along an eyelash line.

Then, a modification of the eye bag support A will be described.

### Second Embodiment

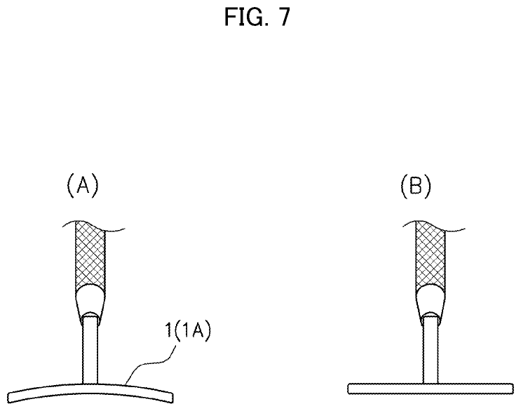

FIG. 7 is a diagram illustrating a curved state of a horizontal receiving part 1, where FIG. 7(A) illustrates a horizontal receiving part 1 in an eye bag support A according to a second embodiment, and FIG. 7(B) illustrates a horizontal receiving part 1 in an eye bag support A according to a first embodiment.

An example in which the horizontal receiving part 1 is curved in a longitudinal direction (left-right direction) is illustrated in FIG. 7(A).

The face of a person is a curved surface in the left-right direction, and a portion of an eye bag is also a slightly curved surface.

Therefore, the horizontal receiving part 1 can more accurately makes contact along the eye bag when curved in the left-right direction, as illustrated in FIG. 7(A).

### Third Embodiment

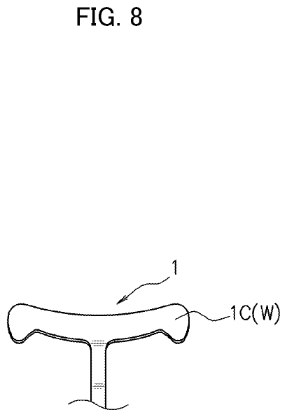

FIG. 8 is a front view illustrating an eye bag support A according to a third embodiment of the present invention.

An example is illustrated in which an extended part W is formed on the end portion side of a horizontal receiving part 1.

The extended part W contacts a skin surface in a wide area, whereby a burden is not easily imposed on an eye bag.

That is, due to the presence of the extended part W, the horizontal receiving part 1 presses the skin surface in a wide area without the end portion side thereof concentrically contacting the skin surface.

Therefore, the eye bag is supported more averagely without any deviation.

### Fourth Embodiment

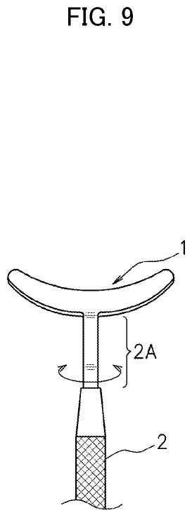

FIG. 9 is a perspective view illustrating an eye bag support A according to a fourth embodiment of the present invention.

An example is illustrated in which a horizontal receiving part 1, together with a neck part 2A, turns in a horizontal direction with respect to a large diameter portion of a grip body 2B.

In this case, the grip part 2 has a freely fitted structure (not illustrated) in which its inner portion is rotatable.

Even if the large diameter portion of the grip body 2B is turned, the horizontal receiving part 1 does not turn.

There is an advantage that even if the grip body 2B turns because a hand inadvertently moves with an eye bag supported, the eye bag can always be supported in the same state.

The eye bag can be evenly supported from below in a balanced manner by only grasping and pressing the grip part 2.

### Fifth Embodiment

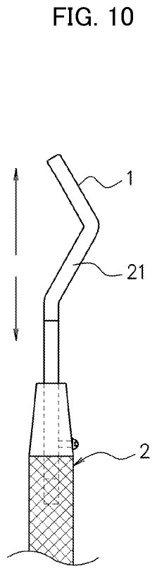

FIG. 10 is a perspective view illustrating an eye bag support A according to a fifth embodiment of the present invention.

An example is illustrated in which a horizontal receiving part 1 can adjust its degree of protrusion from a distal end of a grip body 2B.

A neck part 2A is extended downward, and is removably inserted into the grip body 2B.

Therefore, such an extension can be fixed to a free position with a fastener such as a screw. Accordingly, the degree of protrusion of the horizontal receiving part 1 can be adjusted.

### Sixth Embodiment

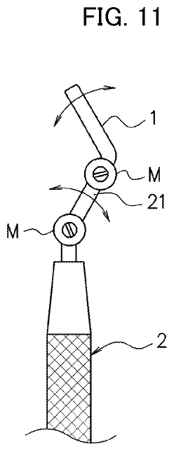

FIG. 11 is a perspective view illustrating an eye bag support A according to a sixth embodiment of the present invention.

An example is illustrated in which a degree of inclination θ1 of an inclined surface 11 and a degree of inclination θ2 of a neck inclined part can be changed.

In other words, an example is illustrated in which a degree of inclination θ1 of a horizontal receiving part 1 with respect to a neck part 2A (specifically, a neck inclined part 21) and a degree of inclination θ2 of the neck part 2A (specifically, the neck inclined part 21) with respect to an axis of a grip body 2B can be each changed (see FIG. 3).

In this case, the neck part 2A is provided with an angle adjustment part M (e.g., an angle adjustment part M capable of adjusting an angle by tightening a screw).

When the angle adjustment part M is provided in the neck part 2A immediately below the horizontal receiving part 1, the angle (the degree of inclination θ1) of the horizontal receiving part 1 with respect to the neck part 2A can be adjusted.

When the angle adjustment part M is provided immediately above the grip body 2B, the angle (the degree of inclination θ2) of the neck part 2A with respect to the grip body 2B can be adjusted.

When the neck part 2A is provided with two angle adjustment parts M vertically spaced apart from each other, the angle of the horizontal receiving part 1 with respect to the neck part 2A can be adjusted, and the angle of the neck part 2A with respect to the axis of the grip body 2B can be adjusted.

In this case, the degree of inclination θ1 and the degree of inclination θ2 can be each separately adjusted, whereby a degree of freedom of a shape further increases, resulting in an eye bag support A accurate for each patient.

Although the preferred embodiments of the present invention have been described above, the present invention is not limited to the above-described embodiments.

As a material for the eye bag support A, for example, hard plastic can also be adopted in addition to a metal such as stainless steel (SUS) or titanium.

Partially different materials can also be used. For example, the horizontal receiving part 1 may be made of a metal, and the grip part 2 may be made of synthetic resin.

Conversely, the horizontal receiving part 1 may be made of synthetic resin, and the grip part 2 may be made of a metal.

In such a combination, the entire eye bag support A can be integrated by insert injection molding of a metal and synthetic resin, for example, as a manufacturing method.

Further, the eye bag support A according to the present invention is of course applicable to not only a procedure for forming an eye bag but also an action for stretching the skin of upper and lower eyelids, for example, during a procedure for applying permanent makeup to the upper and lower eyelids, for example.

In the case, the degrees of inclination θ1 and 02 can be used in a range different in angle from those described above.

### INDUSTRIAL APPLICABILITY

An eye bag support A according to the present invention includes a horizontal receiving part 1 to be brought into contact with an eye bag and a grip part 2 to be grasped, the grip part 2 being formed to hang from the center of the horizontal receiving part 1.

Therefore, in a cosmetic plastic procedure, the eye bag support A is widely applicable to any portion requiring an operation for grasping the grip part 2 and pressing the horizontal receiving part 1 against the skin.

### REFERENCE SIGNS LIST

A . . . eye bag support,
W . . . extended part,
M . . . angle adjustment part,
θ1, θ2 . . . degree of inclination,
1 . . . horizontal receiving part,
11 . . . inclined surface,
1A . . . upper side arc,
1B . . . lower side arc,
1C . . . end portion,
2 . . . grip part,
2A . . . neck part,
2B . . . grip body, and
21 . . . neck inclined part

1. A T-shaped support for upwardly supporting an eye bag of an eye, the eye bag support comprising a horizontal receiving part to be brought into contact with the eye bag and a grip part to be grasped, the grip part being formed to hang from the center of the horizontal receiving part.

2. The eye bag support according to claim 1, wherein the horizontal receiving part is formed in a crescent shape and has both ends each formed into a curve.

3. The eye bag support according to claim 1, wherein the horizontal receiving part and the grip part are each formed of a metal, and the horizontal receiving part is coated with silicone resin.

4. The eye bag support according to claim 1, wherein the grip part includes a neck part having a neck inclined part inclined rearward with respect to an axis provided in its upper portion, and a degree of inclination θ2 of the neck inclined part is 120 degrees to 170 degrees.

5. The eye bag support according to claim 4, wherein the horizontal receiving part is inclined forward with respect to the neck inclined part to form an inclined surface, and a degree of inclination θ1 of the inclined surface is 110 degrees to 160 degrees.

6. The eye bag support according to claim 1, wherein the grip part has a rod shape and has fine irregularities for slippage prevention formed therein.

7. The eye bag support according to claim 5, wherein the neck part is provided with two angle adjustment parts vertically spaced apart from each other, and the degree of inclination θ1 of the inclined surface and the degree of inclination θ2 of the neck inclined part can each be changed.
